(19) United States
(12) Patent Application Publication
(10) Pub. No.: US 2013/0302305 A1
(43) Pub. Date: Nov. 14, 2013

(54) Methods for Treating Gout in Patients Subpopulations

(71) Applicants: Gopal Chandra Saha, Dublin, CA (US); Brian K. Roberts, Palo Alto, CA (US); Brian Edward Lavan, San Francisco, CA (US); Charles A. McWherter, Oakland, CA (US)

(72) Inventors: Gopal Chandra Saha, Dublin, CA (US); Brian K. Roberts, Palo Alto, CA (US); Brian Edward Lavan, San Francisco, CA (US); Charles A. McWherter, Oakland, CA (US)

(73) Assignee: Metabolex, Inc.

(21) Appl. No.: 13/668,024

(22) Filed: Nov. 2, 2012

Related U.S. Application Data
(60) Provisional application No. 61/556,028, filed on Nov. 4, 2011.

(51) Int. Cl. A61K 31/216; A61K 45/06

(52) U.S. Cl. CPC A61K 31/216; A61K 45/06
USPC 424/94.4; 514/534; 514/365; 514/171; 514/383; 514/9.7

(57) ABSTRACT

The present application discloses a method of lowering serum uric acid level in a subject with impaired renal function, comprising administering to the subject a compound of Formula (I), as disclosed herein.

### CROSS-REFERENCE TO RELATED APPLICATIONS

This application claims priority to U.S. Provisional Application No. 61/556,028, filed on Nov. 4, 2011, the complete disclosure of which is incorporated by reference herein.

### BACKGROUND

Conditions associated with elevated serum uric acid levels (hyperuricemia) include disorders of urate crystal deposition such as gout arthropathy and tophi, urolithiasis (urinary tract stones), urate nephropathy, as well as the sequelae of these disorders. Hyperuricemia is associated with an increased risk of developing gout arthropathy, and the risk of gout increases with the degree and duration of the hyperuricemia. In addition to gout arthropathy, chronic hyperuricemia may lead to the deposition of uric acid crystals in the urinary tract, renal parenchyma, and soft tissues, resulting in urolithiasis, urate nephropathy with chronic kidney disease, and soft tissue tophi, respectively.

People with hyperuricemia or gout often have impaired renal function, for example, acute or chronic kidney disease, or may be on aspirin or diuretic therapy. Because of limitations and disadvantages of current urate-lowering agents in such subjects, more effective methods, compositions and therapies to lower uric acid in these populations are needed.

### SUMMARY

In one embodiment, this application is directed to methods of lowering the serum uric acid level in a subject, methods of treating a subject having a condition associated with an elevated serum uric acid levels, and methods for the treatment of hyperuricemia in a subject with gout, wherein the subject is a member of one or more subpopulations, the subpopulations comprising subjects with impaired renal function, subjects on aspirin therapy, and subjects on diuretic therapy.

One aspect provides for a method of lowering serum uric acid level in a subject with impaired renal function, comprising administering to the subject a compound of Formula (I)

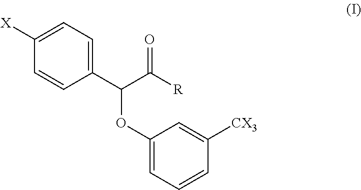

wherein R is selected from the group consisting of hydroxy, lower aralkoxy, di-lower alkylamino-lower alkoxy, lower alkanamido-lower alkoxy, benzamido-lower alkoxy, ureido-lower alkoxy, N′-lower alkyl-ureido-lower alkoxy, carbamoyl-lower alkoxy, halophenoxy-substituted lower alkoxy, carbamoyl-substituted phenoxy, carbonyl-lower alkylamino, N,N-di-lower alkylamino-lower alkylamino, halo-substituted lower alkylamino, hydroxyl-substituted lower alkylamino, lower alkanolyloxy-substituted lower alkylamino, ureido and lower alkoxycarbonylamino; and each X is independently a halogen; or a pharmaceutically acceptable salt thereof.

Other aspects provide methods for treating a subject having a condition associated with an elevated serum uric acid level and with impaired renal function comprising administering to the subject a compound of Formula (I). Other aspects provide methods for the treatment of hyperuricemia in a subject with gout and impaired renal function comprising administering to the subject in need thereof a compound of Formula (I). Other aspects provide methods for the treatment of hyperuricemia in a subject with gout comprising administering to the subject in need thereof a compound of Formula (I), wherein the subject is undergoing aspirin or a diuretic therapy. In some aspects, the compound of Formula (I) is (−)-halofenate, (−)-halofenic acid, or a pharmaceutically acceptable salt thereof. In some aspects, the impaired renal function is chronic kidney disease, and in further aspects the chronic kidney disease is mild or moderate. Further aspects are provided below.

Currently available urate-lowering agents have limitations in their ability to lower serum uric acid to a desirable level in patients with impaired renal function, and their use in this population may be limited by various adverse side effects or toxicities. Therapeutic agents currently in development may have similar limitations. For example, persons with decreased or impaired renal function require lower doses of allopurinol than those with normal renal function, and such persons should be observed closely during the early stages of administration of this drug. (See prescribing information for allopurinol, available at http://www.drugs.com/pro/allopurinol.html). Also, probenecid, a uricosuric, is contraindicated in persons with uric acid kidney stones and loses its effectiveness in persons with impaired renal function. (See prescribing information for probenecid, available at http://www.drugs.com/pro/probenecid.html). Aspirin and diuretics are known in increase serum uric acid, and therefore would counteract the beneficial effects of most serum-urate lowering therapies. Accordingly, advantages of the methods disclosed herein include improved therapeutic benefits, dosing levels, patient monitoring and compliance, and safety profiles in these population over currently available uric-acid lowering therapies or other uric-acid lowering agents in development.

### BRIEF DESCRIPTION OF THE DRAWINGS

FIG. 1 is a chart showing the lowering of serum uric acid (sUA) in human subjects having (a) normal and stage 1 CKD, (b) stage 2 CKD, and (c) stage 3 CKD after treatment with (−)-halofenate at 600 mg per day.

FIG. 2 is a chart showing the lowering of serum uric acid in human subjects having stage 2 and 3 CKD after treatment with (−)-halofenate at dosages of at 200 mg, 400 mg, and 600 mg per day.

FIG. 3 is a scatter plot of change in serum uric acid level in human subjects having stage 3, stage 2 and stage 1 CKD after treatment with (−)-halofenate.

FIG. 4 is a chart showing the change in CrCL in human subjects after treatment with (−)-halofenate at dosages of at 200 mg, 400 mg, and 600 mg per day.

FIG. 5 is a chart showing changes in HbA1c levels in human subjects having stage 2 and stage 3 CKD after treatment with (−)-halofenate.

FIG. 6 is a chart showing changes in fasting plasma glucose (FPG) levels in human subjects having stage 2 and stage 3 CKD after treatment with (−)-halofenate.

FIG. 7 is a chart showing changes in triglyceride (TG) levels in human subjects having stage 2 and stage 3 CKD after treatment with (−)-halofenate.

FIG. 8 is a chart showing changes in serum uric acid levels in human subjects on low- or medium-dose aspirin therapy after treatment with (−)-halofenate.

FIG. 9 is a chart showing changes in serum uric acid levels in human subjects on diuretic therapy after treatment with (−)-halofenate.

### DETAILED DESCRIPTION

As utilized in accordance with the present disclosure, the following terms, unless otherwise indicated, shall be understood to have the following meanings:

“About” when qualifying a number, refers to a range of plus or minus 10 percent of that value or number, unless indicated otherwise. Without limiting the application of the doctrine of equivalents as to the scope of the claims, each number should be construed in light of such factors as the number of reported significant digits and the manner or method (e.g. instrumentation, sample preparation, etc.) used to obtain that number.

“Administering” or “administration” refers to the act of giving a drug, prodrug, or therapeutic agent to a subject. Exemplary routes of administration are discussed below.

“Acute gout” refers to gout present in a subject with at least one gouty symptom (e.g., podagra or other gouty arthritis, gout flare, gouty attack).

“Arhalofenate” refers to (−)-halofenate, i.e. (−)-(R)-(4-chloro-phenyl)-(3-trifluoromethyl-phenoxy)-acetic acid 2-acetylamino-ethyl ester.

“Chronic kidney disease” (“CKD”) refers to the slow loss of kidney function (renal impairment) over time. Kidney (or renal) function may be measured by the glomerular filtration rate (“GFR”), which may be approximated by measuring the creatinine clearance rate (CCr or CrCl) (the volume of blood plasma that is cleared of creatinine per unit time). Creatinine clearance (glomerular filtration rate) can also be estimated using the Cockcroft-Gault equation [Cockcroft-Gault GFR=(140−age)×(Body weight at baseline in kg)×(0.85 if female)/(72×serum creatinine at baseline)]. The CKD groups, or stages of CKD, used herein are defined as follows: CKD0 (normal kidney function)—GFR greater than or equal to 120 mL/min; CKD1—GFR of 90 to 119 mL/min; CKD2 (mild CKD)—GFR of 60 to 89 mL/min; CKD3 (moderate CKD)—GFR of 30 to 59 mL/min; CKD4 (severe CKD)—GFR of 15 to 29 mL/min; CKD5 (kidney failure)—GFR less than 15 mL/min

“Chronic gout” refers to gout present in a subject having recurrent or prolonged gout flares, tophus formation, chronic inflammatory arthritis, or joint deterioration associated with gout, and includes the periods following recovery from acute gout and between acute gout attacks (i.e. intercritical gout).

“Composition” or, interchangeably, “formulation” refers to a preparation that contains a mixture of various excipients and key ingredients that provide a relatively stable, desirable, and useful form of a compound or drug.

The prefixes “d” and “l” or (+) and (−) are employed to designate the sign of rotation of plane-polarized light by the compound, with (+) or d- meaning that the compound is “dextrorotatory” and with (−) or l- meaning that the compound is “levorotatory”. For a given chemical structure, these isomers or “optical isomers” are identical except that they are mirror images of one another. In describing an optically active compound, the prefixes R and S are used to denote the absolute configuration of the molecule about its chiral center(s). There is no correlation between the nomenclature for the absolute stereochemistry and for the rotation of an enantiomer (i.e., the R-isomer can also be the l-isomer). A specific optical isomer can also be referred to as an “enantiomer,” and a mixture of such isomers is often called an “enantiomeric” or “racemic” mixture. See, e.g., A. Streitwiesser & C. H. Heathcock, INTRODUCTION TO ORGANIC CHEMISTRY, 2^{nd} Edition, Chapter 7 (MacMillan Publishing Co., U.S.A. 1981). The optical rotation [α]_{D} of (−)-halofenate was measured in methyl alcohol.

“Elevated serum uric acid level” refers to a serum uric acid level greater than normal and, in patients with gout, generally refers to a serum uric acid level greater than or equal to about 6 mg/dL. In some instances, elevated serum uric acid levels are above the mean level in a given population, such as those of a particular gender or age.

“Effective amount” refers to an amount required (i) at least partly to attain the desired response in a subject; (ii) to delay or to prevent the onset of a particular condition being treated in a subject; or (iii) or to inhibit or to prevent the progression of a particular condition being treated in a subject. The effective amount for a particular subject varies depending upon the health and physical condition of the subject to be treated, the taxonomic group of individual to be treated, the degree of protection desired, the formulation of the composition, the assessment of the medical situation, and other relevant factors. It is expected that the amount will fall in a relatively broad range that can be determined through routine trials.

“First urate-lowering agent” refers to a compound of any of Formula (I), (II), (III) or (IV) or a therapeutically acceptable salt or prodrug thereof. For clarity, this term implies no temporal aspect or relationship, e.g. to a second urate-lowering agent.

“Gout” refers to a group of disorders or symptoms most often associated with the accumulation of uric acid due to an overproduction of uric acid or a reduced ability of the kidney to excrete uric acid. Gout is often characterized by the deposition of urate crystals (uric acid or salts thereof, e.g. monosodium urate) in the joints (gouty arthropathy) or soft tissue (tophi). “Gout” as used herein includes acute gout, chronic gout, moderate gout, refractory gout and severe gout.

“Gout-associated inflammation” refers to local or systemic inflammation due to immune responses to the deposition of urate crystals.

“Halofenate” refers to compounds of Formula (III) below, i.e. (4-chlorophenyl)-(3-trifluoromethylphenoxy)-acetic acid 2-acetylaminoethyl ester (also referred to as the 2-acetamidoethyl ester of 4-chlorophenyl-(3-trifluoromethylphenoxy)-acetic acid). The term halofenate and the corresponding chemical names include both the (+) and (−) enantiomer of compounds of Formula (III) as well as mixtures thereof, unless otherwise specified.

“Halofenic acid” and “CPTA” refer to the compounds of Formula (IV), i.e. 4-chlorophenyl-(3-trifluoromethylphenoxy)-acetic acid [also referred to as 2-(4-chlorophenyl)-2-(3-(trifluoromethyl)phenoxy)acetic acid] as well as its pharmaceutically acceptable salts. The term halofenic acid and the corresponding chemical names include both the (+) and (−) enantiomer of compounds of Formula (IV) as well as mixtures thereof, unless otherwise specified.

“Hyperuricemia” refers to an elevated serum uric acid level (see above).

“Impaired renal function” refers to a medical condition in which the kidneys fail to adequately filter toxins and waste products from the blood. Impaired renal function includes acute kidney injury and chronic kidney disease (i.e. CKD1-5).

“Moderate gout” refers to gout present in a subject having at least two gout flares in the past 12 months.

“Pharmaceutically acceptable” refers to that which is useful in preparing a pharmaceutical composition that is generally safe, non-toxic, and neither biologically nor otherwise undesirable, and includes that which is acceptable for veterinary or human pharmaceutical use.

“Pharmaceutically acceptable salt” includes pharmaceutically acceptable acid addition salts and pharmaceutically acceptable base addition salts and includes both solvated and unsolvated forms. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

“Pharmaceutically acceptable acid addition salt” refers to salts formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salt” refers to salts prepared from the addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

“Refractory gout” refers to gout in patients who are unresponsive or poorly responsive to one or more second urate-lowering agents, or have experienced or are at an increased risk of experiencing an adverse event therefrom. The terms “unresponsive” and “poorly responsive” in this context include (1) no or insignificant lowering of serum uric acid, (2) failure to reach a target serum uric acid level (e.g. as determined by a physician or other medical practitioner), and (3) the persistence of one or more gouty conditions or symptoms such as gout flares, gouty tophus, gouty arthritis, or other associated conditions regardless of any lowering of serum uric acid levels.

“Second urate-lowering agent” refers to a therapeutic agent that lowers serum uric acid levels that is not a first urate-lowering agent. Second urate-lowering agents include currently available agents (i.e. an agent approved by the FDA or other appropriate regulatory authority as of the filing date of this application) that lower serum uric acid, as well as compounds currently in development or under regulatory review. Examples of second urate-lowering agents are provided below. For clarity, this term implies no temporal aspect or relationship, e.g. to a first urate-lowering agent.

“Subject” and “patient” refer to animals such as mammals, including humans, other primates, domesticated animals (e.g. dogs, cats), farm animals (e.g. horses, cattle, goats, sheep, pigs), rats and mice.

“Severe gout” refers to gout present in a subject having tophaceous deposits in the joints, skin, or kidneys resulting in chronic arthritis, joint destruction, subcutaneous tophi, or kidney dysfunction, and, in some cases, with subsequent deformity and/or disability.

“Substantially free from” when used in reference to (−)-halofenate or (−)-halofenic acid (or a salt thereof) being substantially free from the corresponding (+) enantiomer (i.e. (+)-halofenate, (+)-halofenic acid, or a salt thereof) refers to a composition containing a high proportion of a compound's (−) enantiomer in relation to the (+) enantiomer. In one embodiment, the term means that by weight, the compound included in the composition is at least 85% (−) enantiomer and at most 15% (+) enantiomer. In one embodiment, the term means that by weight, the compound included in the composition is at least 90% (−) enantiomer and at most 10% (+) enantiomer. In other embodiments, the term means that by weight, the compound included in the composition is at least 91% (−)enantiomer and at most 9% (+) enantiomer, at least 92% (−) enantiomer and at most 8% (+) enantiomer, at least 93% (−) enantiomer and at most 7% (+) enantiomer, at least 94% (−) enantiomer and at most 6% (+) enantiomer, at least 95% (−) enantiomer and at most 5% (+) enantiomer, at least 96% (−) enantiomer and at most 4% (+) enantiomer, at least 97% (−) enantiomer and at most 3% (+) enantiomer, at least 98% (−) enantiomer and at most 2% (+) enantiomer, or at least 99% (−) enantiomer or greater than 99% (−) enantiomer. Other percentages of the (−) and (+) enantiomers may also be provided. These percentages are based upon the amount of the enantiomer relative to the total amount of both enantiomers of the compound in the composition.

“Therapeutically effective dose”, “therapeutically effective amount”, or, interchangeably, “pharmacologically acceptable dose” and “pharmacologically acceptable amount” mean that a sufficient amount of a therapeutic agent, therapeutic agents, or metabolites thereof will be present in order to achieve a desired result, e.g., lowering uric acid levels to a target goal or treating gout in its various forms or treating conditions associated with hyperuricemia.

“Treatment” and “treating” of a disease, disorder, condition or symptom refer to (1) preventing or reducing the risk of developing the disease, disorder or condition, i.e., causing the clinical symptoms of the disease, disorder or condition not to develop in a subject who may be exposed to or predisposed to the disease, disorder or condition but who does not yet experience or display symptoms of the disease, disorder or condition (i.e. prophylaxis); (2) inhibiting the disease, disorder or condition, i.e., arresting or reducing the development of the disease, disorder or condition or its clinical symptoms; and (3) relieving the disease, disorder or condition, i.e., causing regression, reversal, or amelioration of the disease, disorder or condition or reducing the number, frequency, duration or severity of one or more of its clinical symptoms. The term “management” may be used synonymously.

“Urate” refers to uric acid (7,9-dihydro-1H-purine-2,6,8(3H)-trione) and ions and salts thereof.

This application describes methods for lowering the serum uric acid level in a subject comprising administering to a subject in need thereof a compound of Formula (I)

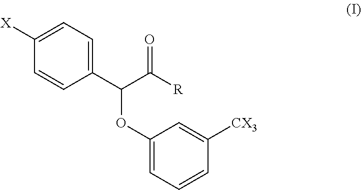

wherein R is selected from the group consisting of a hydroxy, lower aralkoxy, di-lower alkylamino-lower alkoxy, lower alkanamido-lower alkoxy, benzamido-lower alkoxy, ureido-lower alkoxy, N′-lower alkyl-ureido-lower alkoxy, carbamoyl-lower alkoxy, halophenoxy substituted lower alkoxy, carbamoyl substituted phenoxy, carbonyl-lower alkylamino, N,N-di-lower alkylamino-lower alkylamino, halo substituted lower alkylamino, hydroxy substituted lower alkylamino, lower alkanolyloxy substituted lower alkylamino, ureido, and lower alkoxycarbonylamino; and each X is independently a halogen, or a pharmaceutically acceptable salt thereof, wherein the subject has impaired renal function.

In certain aspects, the compound is a compound of Formula (II)

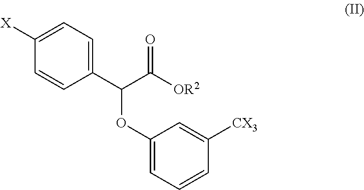

wherein R² is a member selected from the group consisting of phenyl-lower alkyl, lower alkanamido-lower alkyl, and benzamido-lower alkyl; and each X is independently a halogen, or a pharmaceutically acceptable salt thereof.

In other aspects, the compound is a compound of Formula (III), also referred to as halofenate

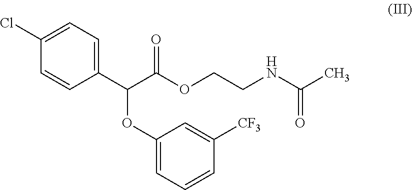 [SMILES: CC(=O)NCCOC(=O)C(Oc1cccc(C(F)(F)F)c1)c1ccc(Cl)cc1]

or a pharmaceutically acceptable salt thereof.

In other aspects, the compound is a compound of Formula (IV), also referred to as halofenic acid

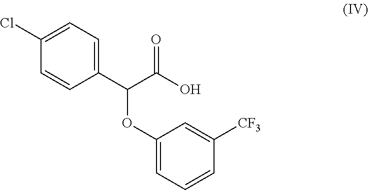 [SMILES: O=C(O)C(Oc1cccc(C(F)(F)F)c1)c1ccc(Cl)cc1]

or a pharmaceutically acceptable salt thereof.

It should be noted that any carbon atom with unsatisfied valences in the formulae and examples herein is assumed to have the hydrogen atom to satisfy the valences.

In certain embodiments the compound is a compound that generates the compound of Formula (IV) or a pharmaceutically acceptable salt thereof via a chemical reaction after being administered, as discussed in more detail below.

In certain embodiments, the compound is the (−) enantiomer of a compound of Formula (I), (II), (III) or (IV). In certain embodiments, the compound is (−)-halofenate (i.e. (−)-(R)-(4-chloro-phenyl)-(3-trifluoromethyl-phenoxy)-acetic acid 2-acetylamino-ethyl ester, also referred to as arhalofenate). In other embodiments, the compound is (−)-halofenic acid (i.e. (−)-4-chlorophenyl-(3-trifluoromethylphenoxy) acetic acid) or a pharmaceutically acceptable salt thereof. In certain embodiments, the (−)-halofenate, (−)-halofenic acid, or pharmaceutically acceptable salt thereof is substantially free from the corresponding (+) enantiomer.

The enantiomers (stereoisomers) of compounds of Formula (I), (II), (III) or (IV) and pharmaceutically acceptable salt thereof can be prepared by using reactants or reagents or catalysts in their single enantiomeric form in the process wherever possible or by resolving the mixture of stereoisomers by conventional methods including use of microbial resolution, resolving the diastereomeric salts formed with chiral acids or chiral bases and chromatography using chiral supports. See, also U.S. Pat. No. 7,199,259 (Daugs), U.S. Pat. Nos. 6,646,004; 6,624,194; 6,613,802; and 6,262,118 (each to Luskey et al.), U.S. Pat. No. 7,714,131 (Zhu et al.), U.S. Pat. No. 7,432,394 (Cheng et al.) and U.S. Publication No. 2010/0093854 (Broggini et al.) each of which are incorporated herein by reference in their entireties.

The chemical synthesis of racemic mixtures of (3-trihalomethylphenoxy) (4-halophenyl) acetic acid derivatives can also be performed by the methods described in U.S. Pat. No. 3,517,050, the teachings of which are incorporated herein by reference. The individual enantiomers can be obtained by resolution of the racemic mixture of enantiomers using conventional means known to and used by those of skill in the art. See, e.g., J. Jaques et al., in ENANTIOMERS, RACEMATES, AND RESOLUTIONS, John Wiley and Sons, New York (1981). Other standard methods of resolution known to those skilled in the art, including but not limited to, simple crystallization and chromatographic resolution, can also be used (see, e.g., STEREOCHEMISTRY OF CARBON COMPOUNDS (1962) E. L. Eliel, McGraw Hill; J. Lochmuller, Chromatography 113, 283-302 (1975)). Additionally, halofenate, halofenic acid, or a pharmaceutically acceptable salt thereof, i.e., the optically pure isomers, can be prepared from the racemic mixture by enzymatic biocatalytic resolution. Enzymatic biocatalytic resolution has been generally described previously (see, e.g., U.S. Pat. Nos. 5,057,427 and 5,077,217, the disclosures of which are incorporated herein by reference). Other generic methods of obtaining enantiomers include stereospecific synthesis (see, e.g., A. J. Li et al., Pharm. Sci. 86, 1073-1077 (1997)).

The present disclosure also provides for methods for treating one or more conditions associated with an elevated serum uric acid level, i.e. hyperuricemia, comprising administering to a subject in need thereof a compound of Formula (I), (II), (III) or (IV) or a pharmaceutically acceptable salt thereof wherein the subject has impaired renal function. Conditions associated with hyperuricemia include, but are not limited to gout; acute gout; chronic gout; moderate gout; refractory gout; severe gout; deposition of uric acid crystals in the urinary tract, renal parenchyma, soft tissues, joints, cartilage or bones; urolithiasis; urate nephropathy; tophi; podagra; acute inflammatory gouty arthritis; joint destruction; urinary tract infections; renal impairment; chronic kidney disease; kidney stones; local inflammation; systemic inflammation; immune-related disorders; cardiovascular disease including peripheral vascular disease, coronary artery disease and cerebrovascular disease; insulin resistance; diabetes; fatty liver disease; dementia including vascular dementia; dyslipidemia; preeclampsia; hypertension; obesity; muscle spasm; localized swelling; pain including joint pain, muscle fatigue; tumor-lysis syndrome; and stress feelings. Additional conditions associated with hyperuricemia include, but are not limited to, Lesch-Nyhan syndrome, Kelley-Seegmiller syndrome and related conditions.

A variety of factors increase the risk that a patient will have gout or will experience one or more of its symptoms. In addition to hyperuricemia, these factors include obesity, diabetes, chronic kidney disease, hypertension, use of diuretic drugs and certain other drugs (e.g. salicylates, pyrazinamide, ethambutol, nicotinic acid, cyclosporin, 2-ethylamino-1,3,4-thiadiazole, fructose and cytotoxic agents), overeating or fasting, a high purine diet, a high fructose diet, exposure to lead, consumption of red meat and protein, alcohol intake, and injury or recent surgery. Acute gout can be precipitated by perioperative ketosis in surgical patients, reduced body temperature, e.g., while sleeping, and by dehydration, e.g., by use of diuretic drugs. Genetic risk factors for gout and hyperuricemia have also been identified.

In various embodiments, the methods described herein may be used to treat any of the aforementioned conditions or disorders. That is, in one embodiment, the condition associated with an elevated serum uric acid level is gout. In some embodiments, the subject has acute gout. In some embodiments, the subject has experienced one or more gout flares. In some embodiments, the subject has chronic gout. In some embodiments the subject has moderate gout. In some embodiments the subject has refractory gout. In some embodiments the subject has severe gout.

Certain methods provide for the treatment of hyperuricemia in a subject with gout. For example, methods provide for the treatment of hyperuricemia in a subject with gout comprising administering a pharmaceutical composition comprising a first urate-lowering agent, wherein the subject has impaired renal function. In certain methods, the compound can be (−)-halofenate, (−)-halofenic acid, or a pharmaceutically acceptable salts thereof. In certain embodiments, the treatment can be for about four weeks or longer, for about one month or longer, for about 12 week or longer, for about three months or longer, for about six months or longer, for about one year or longer, for about two years or longer, for about five years or longer, or for about 10 years or longer. In certain embodiments the treatment can be indefinite, e.g. for the remainder of the lifetime of the subject.

In various embodiments, the methods comprise treating gout. In some embodiments, the methods comprise treating gout by preventing gout flares. In another embodiment the method comprises reducing the number, frequency, duration or severity of one of more gout flares. In another embodiment the method comprises preventing, reducing or reversing uric acid crystal formation. In some embodiments of the methods for treating uric acid crystal formation, the uric acid crystal formation is in one or more of the joints, under skin, and kidney. In some embodiments, the formations include tophaceous deposits. In some embodiments, the subject has uric acid crystal formation determined by aspiration of tophi or by aspiration of synovial fluid of an inflamed joint. In another embodiment the method comprises reducing uric acid burden. In another embodiment the method comprises reducing the size or number of tophi. The size or number of tophi may be assessed by known methods, for example, use of CT scans.

In various embodiments, the methods described herein lower serum uric acid levels in a subject by about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more, as compared to serum uric acid levels in the subject prior to administering the methods described herein. In various embodiments, serum uric acid levels are decreased about 5% to about 50%, decreased by about 25% to about 75%, or decreased by about 50% to about 99%. Methods to determine serum uric acid levels are well known in the art and are often measured as part of a standard chemistry panel of blood serum samples.

In some embodiments, the methods of the present disclosure lower serum uric acid levels in a subject to about 7 mg/dL or less, to about 6.5 mg/dL or less, to about 6 mg/dL or less, to about 5 mg/dL or less, to about 4 mg/dL or less, or to about 3 mg/dL or less as compared to serum uric acid levels in the subject prior to administering the methods or compositions described herein. In some embodiments, the methods of the present disclosure lower serum uric acid levels in a subject by 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mg/dL, or greater, as compared to serum uric acid levels in the subject prior to administering the methods or compositions described herein. In further embodiments, the methods described herein lower serum uric acid levels by between 0.1 and 10.0 mg/dL, between 0.5 and 6.0 mg/dL, between 1.0 and 4.0 mg/dL or between 1.5 and 2.5 mg/dL. The appropriate serum uric acid level may vary depending on the subject, and may vary for a given subject over time, depending upon the subject's overall medical condition. Similarly, the appropriate serum uric acid level for one group of subjects sharing a common medical condition may be different from that which is appropriate for a different group of subjects sharing a different medical condition. Thus, it may be advisable to reduce the serum uric acid level of a given group of subjects to, for example, below about 5 mg/dL, and to reduce the serum uric acid level of a different group of subjects to, for example, below about 4 mg/dL. In certain embodiments, the methods of the present disclosure decrease a serum uric acid level in the subject by an amount sufficient to result in the disappearance, reduction, amelioration, or the prevention of the onset of one or more conditions associated with elevated serum uric acid over a certain timeframe, for example for about four weeks or longer, for about one month or longer, for about 12 week or longer, for about three months or longer, for about six months or longer, for about one year or longer, for about two years or longer, for about five years or longer, or for about 10 years or longer. For example, a method can decrease the serum uric acid level in a subject by an amount sufficient to result in the disappearance or reduction of tophi over about one week, about one month, about six months, about one year, about two years, or longer, e.g. indefinitely, e.g. for the remainder of the lifetime of the subject.

In further embodiments, the methods of the present disclosure comprise administering a pharmaceutical composition comprising a first urate-lowering agent to a subject whose serum uric acid level is at least about 4 mg/dL, at least about 5 mg/dL, at least about 6 mg/dL, at least about 6.8 mg/dL, at least about 7 mg/dL, at least about 8 mg/dL, at least about 9 mg/dL, at least about 10 mg/dL, or at least about 11 mg/dL. The amount of decrease of serum uric acid level that is appropriate may vary depending on the subject, depending upon the subject's overall medical condition. Similarly, the amount of decrease of serum uric acid level that is appropriate for one group of subjects sharing a common medical condition may be different from that which is appropriate for a different group of subjects sharing a different medical condition.

As described above, certain subjects of the methods described herein have impaired renal function, for example chronic kidney disease (CKD). In some embodiments, subjects will have a certain non-normal stage of CKD as defined above, e.g. CKD1, CKD2 (mild), CKD3 (moderate), CKD4 (severe), or CKD5 (kidney failure). A subject's CKD stage may change over time. In other embodiments, the subject has another type of impaired renal function, e.g. acute kidney injury.

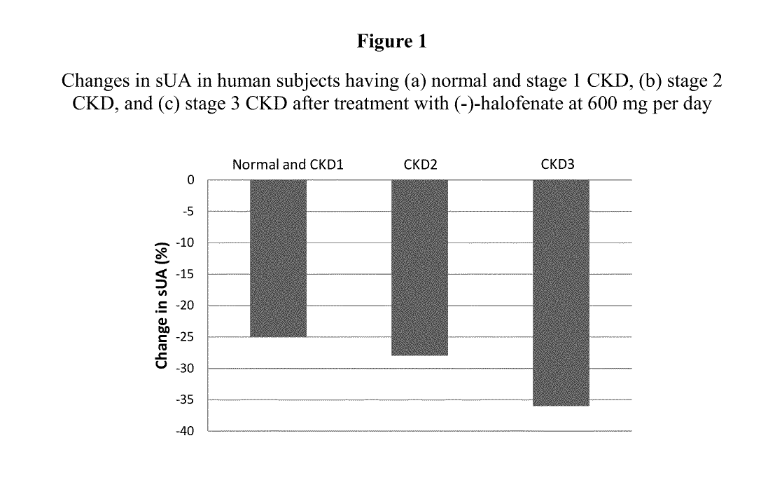

As shown in FIG. 1, serum uric acid can be lowered in patients having (a) normal and stage 1 CKD, (b) stage 2 CKD, and (c) stage 3 CKD after treatment with (−)-halofenate at 600 mg per day. These data suggest that at certain doses of (−)-halofenate (e.g. 600 mg day), the effect of lowering serum uric acid may be more pronounced in patients with more severe CKD. Thus, in certain embodiments, the compound is administered at a dose depending on the stage of CKD of the patient. For example, the dose of a compound described herein which is recommended to subjects having normal kidney function can be reduced in subjects having non-normal CKD, for example in those patients with CKD3 (a GFR between 30 and 59 mL/min) or in patients with CKD4-5 (a GFR lower than 30 mL/min).

In embodiments, methods may comprise measuring the renal function of a subject. For example, certain methods may comprise measuring the renal function of a subject before administering a compound of Formula (I), (II), (III) or (IV) or a pharmaceutically acceptable salt thereof, and certain methods may comprise measuring the renal function of the subject after a certain duration of treatment, e.g. after about 1 day, after about 1 week, after about 2 week, after about 1 month, after about 3 months, after about 6 months or after about 1 year. In certain methods the renal function of the subject may be monitored by taking two or more successive renal function measurements. For example, a first renal function measurement can be taken at or shortly before a compound of the present disclosure is first administered (e.g. before the subject procures the compound, several hours before, one or two days before, etc.), and a second renal function measurement can be taken after a certain duration of treatment as above (e.g. after about 1 week, after about 1 month, etc.). In some methods the subject will have the same degree of renal impairment (e.g. have the same stage of CKD, i.e. CKD1, CKD2, etc.) from the first renal function measurement to the second renal function measurement. In other embodiments the subject's renal impairment will have improved from the first renal function measurement to the second renal function measurement (e.g. the subject can have CKD3 at the first measurement and CKD2 at the second measurement, etc.). Renal function may be measured by GFR or by other methods known to those with ordinary skill in the art.

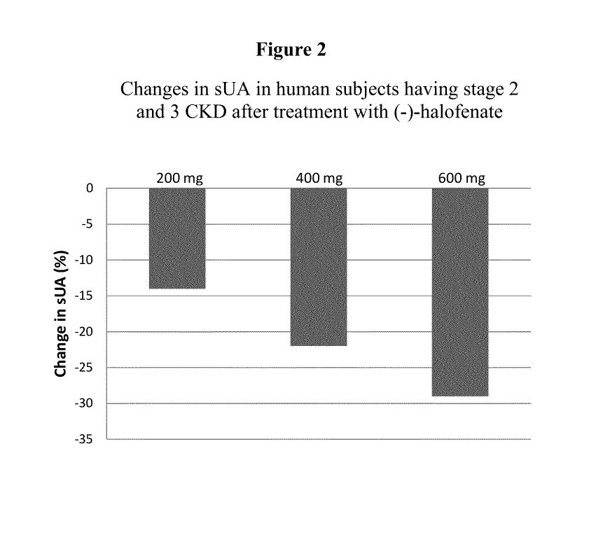

FIG. 2 shows a dose-dependent lowering of serum uric acid in subjects with stage 2 and stage 3 CKD after treatment with (−)-halofenate at dosages of at 200 mg, 400 mg, and 600 mg per day. The data show lowering of serum uric acid in patients having mild to moderate CKD over a broad dose range comparable to the lowering of serum uric acid in patients having normal kidney function.

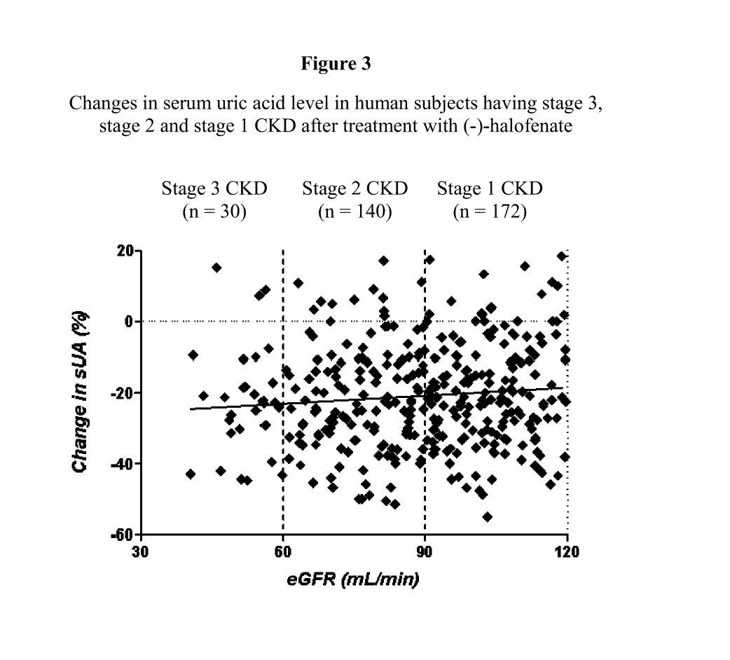

In certain embodiments, the compound is administered at a dose that is independent of the stage of chronic kidney disease. For example, and in contrast to certain second urate-lowering agents such as probenecid and allopurinol, in certain embodiments the dose of compounds described herein is not adjusted or does not have to be adjusted when administered to patients having mild, moderate, or severe CKD if comparable serum-acid lowering is desired. FIG. 3 shows lowering of serum uric acid levels in human subjects having stage 3, stage 2 and stage 1 CKD after treatment with (−)-halofenate. The data demonstrate that the administration of (−)-halofenate results in a lowering of serum uric acid in patients having CKD0, CKD1, CKD2, and CKD3 (normal kidney function to moderate CKD) that is independent of the stage of CKD.

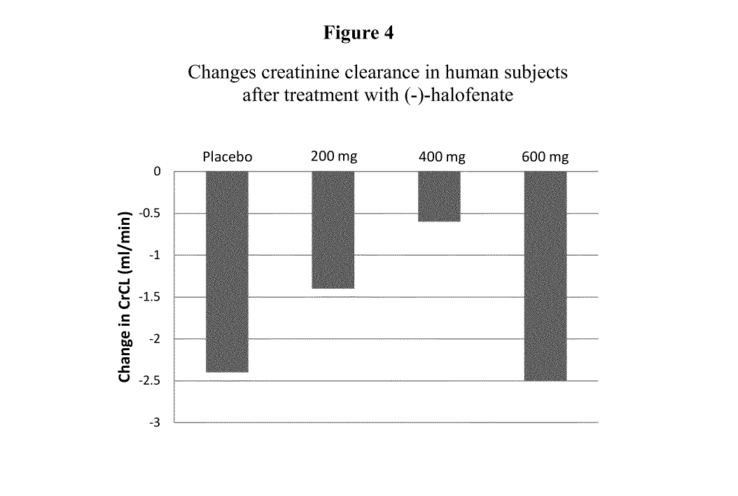

In certain embodiments, the administration of a first urate-lowering agent may comprise the resulting in no clinically significant adverse effect on kidney function. For example, methods include a method for the treatment of hyperuricemia in a subject with gout, the method comprising administering a pharmaceutical composition comprising a first urate-lowering agent, wherein the subject has impaired renal function and wherein the administration results in no clinically significant adverse effect on kidney function. FIG. 4 shows changes in CrCL in human subjects after treatment with (−)-halofenate at dosages of at 200 mg, 400 mg, and 600 mg per day. As described above, creatinine clearance can be used to measure kidney function. As shown in FIG. 4, the administration of (−)-halofenate resulted in no clinically significant change in CrCL as compared to placebo, indicating that (−)-halofenate does not worsen kidney function at the tested doses and durations of administration.

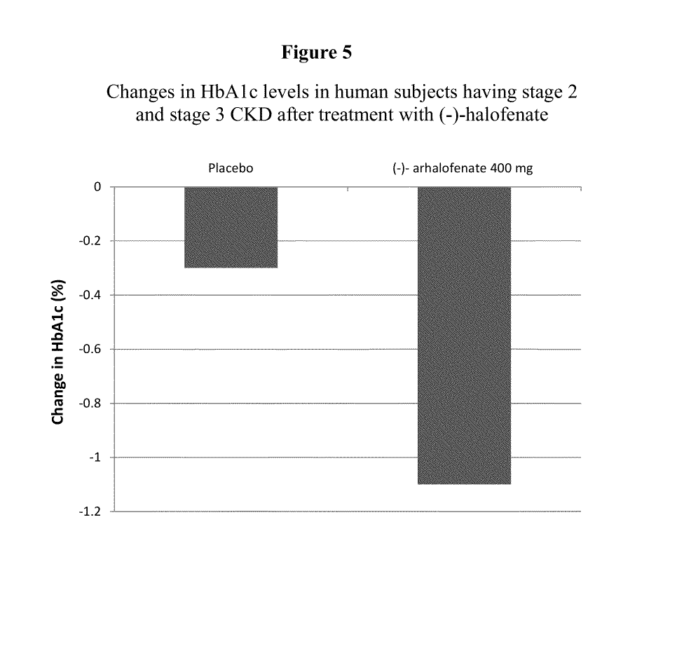

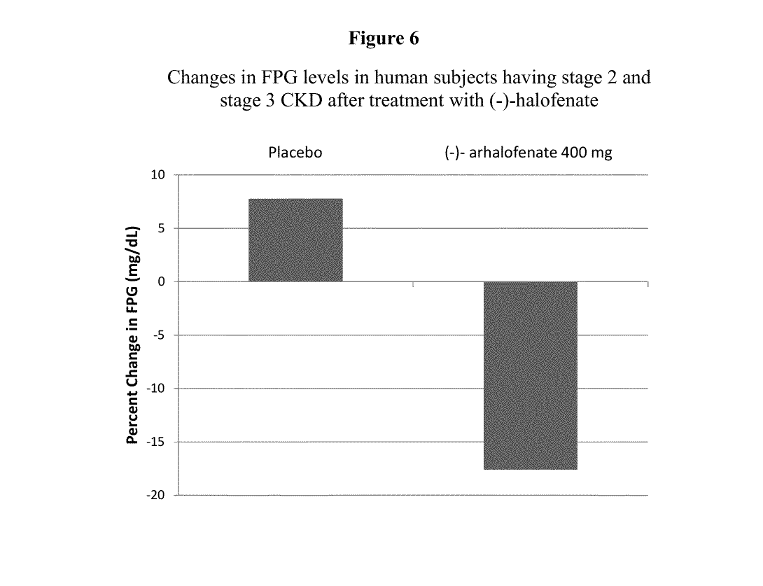

This application also provides for methods of treating hyperuricemia or a condition associated with hyperuricemia comprising administering a first urate-lowering agent (i.e. a compound of Formula (I), (II), (III) or (IV) or a pharmaceutically acceptable salt thereof) wherein the administration results in a lowering of HbA1c, blood glucose, or fasting plasma glucose levels. HbA1c or hemoglobin A1c comprises the main portion of glycosylated hemoglobin in the blood. The ratio of glycosylated hemoglobin to total hemoglobin is proportional to blood glucose levels. Thus, levels of HbA1c serve as markers of blood glucose. FIG. 5 shows changes in HbA1c levels in human subjects having stage 2 and stage 3 CKD after treatment with (−)-halofenate, and FIG. 6 shows similar changes in fasting plasma glucose. In certain methods the compound is (−)-halofenate. In certain methods the condition associated with hyperuricemia is gout. In certain methods the subject has an elevated fasting plasma glucose level (i.e. a level above 100 mg/dL; see, e.g., A. Tirosh et al., N. Engl. J. Med., 353, 1454-62 (2005)). In certain methods the administration of the compound results in a lowering of HbA1c levels by at least about 0.6%, by at least about 0.7%, by at least about 0.8%, by at least about 0.9%, by at least about 1.0%, by at least about 1.1%, by at least about 1.2%, by at least about 1.3%, by at least about 1.4%, by at least about 1.5%, by at least about 1.6%, by at least about 1.7%, by at least about 1.8%, by at least about 1.9%, or by at least about 2.0%. In certain methods the administration of the compound results in a lowering of blood glucose or fasting plasma glucose levels by at least about 10%, by at least about 11%, by at least about 12%, by at least about 13%, by at least about 14%, by at least about 15%, by at least about 16%, by at least about 17%, by at least about 18%, by at least about 19%, or by at least about 20%. In certain of these methods, the compound is administered to the subject once per day for about four weeks or longer, for about one month or longer, for about three months or longer, for about six months or longer, for about one year or longer, for about five years or longer, or indefinitely.

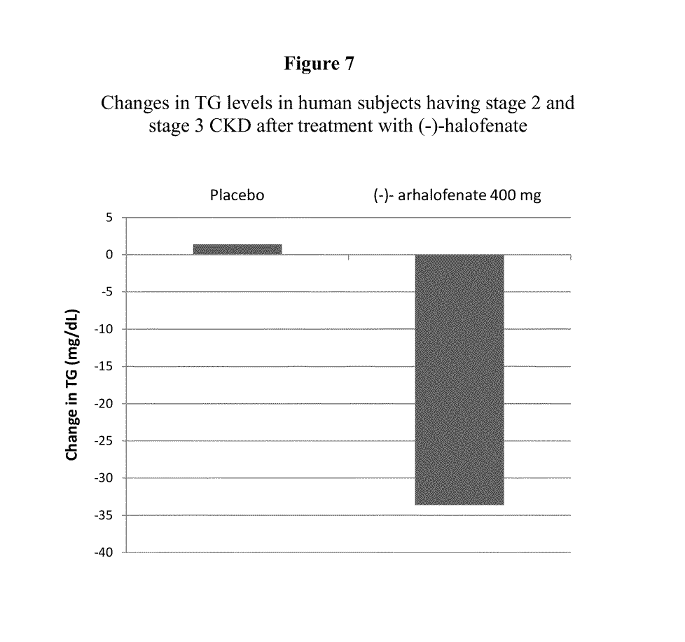

This application also provides for methods of treating hyperuricemia or a condition associated with hyperuricemia comprising administering to a subject in need thereof a first urate-lowering agent, wherein the subject has impaired renal function and wherein the administration results in a lowering of triglyceride levels. FIG. 7 shows changes in triglyceride levels in human subjects having stage 2 and stage 3 CKD after treatment with (−)-halofenate. In certain methods the compound is (−)-halofenate. In certain methods the condition associated with hyperuricemia is gout. In certain methods the subject has an elevated triglyceride level (i.e. a level above 150 mg/dL; see, e.g., A. Tirosh et al. supra). In certain methods the administration of the compound results in a lowering of triglyceride levels by at least about 20%, e.g. by at least about 30%. In certain of these methods, the compound is administered to the subject once per day for about four weeks or longer, for about one month or longer, etc.

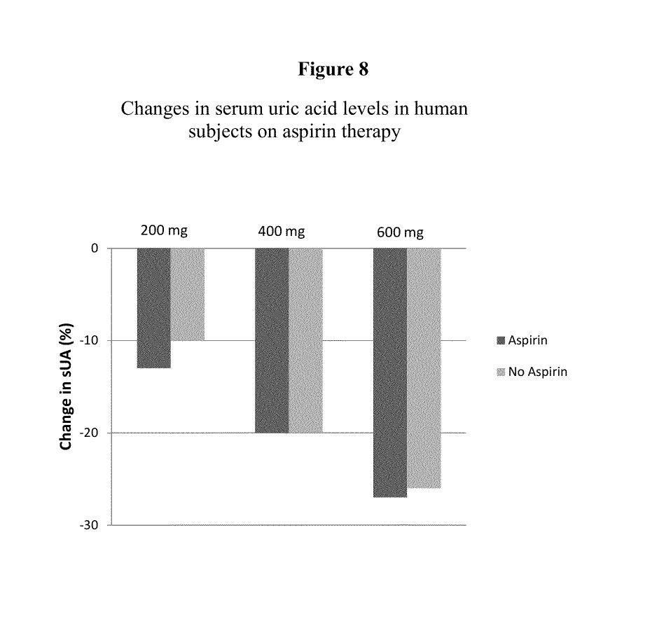

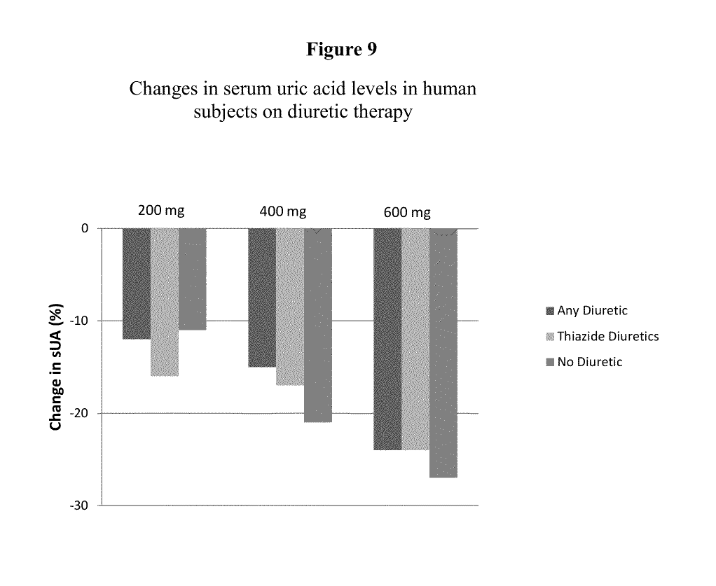

This application also provides for methods of (1) lowering the serum uric acid level in a subject; (2) treating a subject having a condition associated with an elevated serum uric acid levels; and (3) treating hyperuricemia in a subject with gout, the methods comprising administering to a subject in need thereof a compound of Formula (I), (II), (III) or (IV) or a pharmaceutically acceptable salt thereof, wherein the subject is a member of one or more subpopulations, the subpopulations comprising subjects on aspirin therapy and subjects on diuretic therapy (i.e. receiving or being administered aspirin or a diuretic). In certain embodiments the subject is on aspirin therapy at a low or medium dose (e.g. at or less than 325 mg/day). Exemplary diuretics include, but are not limited to high-ceiling or loop diuretics such as ethacrynic acid, torsemide and bumetanide, low-ceiling diuretics, thiazides such as hydrochlorothiazide, carbonic anhydrase inhibitors such as acetazolamide and methazolamide, potassium-sparing diuretics such as spironolactone, potassium canreonate, amiloride and triamterene, calcium-sparing diuretics and osmotic diuretics such as mannitol. FIG. 8 is a chart showing lowering of serum uric acid levels in human subjects on low- or medium-dose aspirin therapy. FIG. 9 is a chart showing lowering of serum uric acid levels in human subjects on diuretic therapy.

The methods described herein may be useful in subjects with refractory gout. Subjects with refractory gout are unresponsive or poorly responsive to one or more second urate-lowering agents, or have experienced or are at an increased risk of experiencing an adverse event therefrom.

The second urate-lowering agent may be any agent that lowers serum uric acid levels that is not a first urate-lowering agent (i.e. not a compound of any of Formula (I), (II), (III) or (IV) or a pharmaceutically acceptable salt thereof). These second urate-lowering agents include inhibitors of uric acid production (e.g. xanthine oxidase inhibitors and purine nucleoside phosphorylase inhibitors), uricosuric agents, and uricases. Xanthine oxidase inhibitors include, but are not limited to: allopurinol, febuxostat, oxypurinol, tisopurine, an inositol and propolis. In some embodiments, the xanthine oxidase inhibitor is allopurinol, febuxostat, oxypurinol, tisopurine, inositol, phytic acid, myo-inositiol, kaempferol, myricetin, and quercetin. Allopurinol (1,5-dihydro-4H-pyrazolo[3,4-d]pyrimidin-4-one), a xanthine oxidase inhibitor, is the current first line standard of care for lowering urate levels. Another xanthine oxidase inhibitor, febuxostat (2-(3-cyano-4-isobutoxyphenyl)-4-methyl-1,3-thiazole-5-carboxylic acid), was approved for treatment of gout in February 2009. Purine nucleoside phosphorylase (PNP) inhibitors represent a relatively new approach to lowering serum uric acid levels in patient with hyperuricemia, gout and related conditions. In some embodiments, the PNP inhibitor is forodesine (BCX1777) (BioCryst Pharmaceuticals, Inc.). In other embodiments, the PNP inhibitor is ulodesine (BCX4208; 7-(((3R,4R)-3-hydroxy-4-(hydroxymethyl)pyrrolidin-1-yl)methyl)-3H-pyrrolo[3,2-d]pyrimidin-4(5H)-one) (BioCryst Pharmaceuticals, Inc.). Ulodesine monotherapy administered at 40, 80, 120, 160 and 240 mg/day has been shown to rapidly and significantly reduced serum uric acid in gout patients. Uricosuric agents enhance renal excretion of uric acid and generally act by lowering the absorption of uric acid from the kidney proximal tubule back to the blood, e.g., by inhibiting urate transporters, e.g, SLC22A12. Uricosuric agents include, but are not limited to, probenecid, 2-((5-bromo-4-(4-cyclopropylnaphthalen-1-yl)-4H-1,2,4-triazol-3-yl)thio)acetic acid (RDEA594, lesinurad), potassium 4-(2-((5-bromo-4-(4-cyclopropylnaphthalen-1-yl)-4H-1,2,4-triazol-3-yl)thio)acetamido)-3-chlorobenzoate (RDEA806), RDEA684, benzbromarone, sulfinpyrazone, amlodipine, atorvastatin, fenofibrate, levotofisopam, guaifenesin, losartan, adrenocorticotropic hormone and cortisone. Probenecid is the most commonly used uricosuric agent in the U.S. and may be given in combination with allopurinol to some gout patients. Benzbromarone and sulfinpyrazone are also used as first line uricosuric agents. Guaifenesin, losartan, atorvastatin, amlodipine, adrenocorticotropic hormone (ACTH or corticotropin), fenofibrate and cortisone also have uricosuric effects. Uricase or urate oxidase enzymes are found in many mammals but not humans. They can lower uric acid levels by converting uric acid into allantoin, a benign end metabolite which is easily excreted in the urine. Uricase enzymes include, but are not limited to, rasburicase or a pegylated uricase enzyme (PEG-uricase). In some embodiments, the pegylated uricase enzyme is Krystexxa® (PURICASE®; pegloticase) (Savient Pharmaceuticals, Inc.) which is approved in the U.S. for the treatment of chronic gout in adult patients refractory to conventional therapy.

In certain embodiments, the subject is refractory to allopurinol, 2-((5-bromo-4-(4-cyclopropylnaphthalen-1-yl)-4H-1,2,4-triazol-3-yl)thio)acetic acid (RDEA594, lesinurad), 2-(3-cyano-4-isobutoxyphenyl)-4-methyl-1,3-thiazole-5-carboxylic acid (febuxostat), or BCX4208. In some embodiments the subject is refractory to allopurinol. For example, in one embodiment, the subject is refractory to allopurinol administered at from 100 mg/day to 800 mg/day (e.g. from 100 mg/day to 300 mg/day) for about one month or longer, about three months or longer, about one year or longer, etc. In some embodiments the subject is refractory to febuxostat. For example, in one embodiment the subject is refractory to febuxostat administered at from 40 mg/day to 120 mg/day for about one month or longer, about three months or longer, about one year or longer, etc. In certain embodiments the subject has mild or moderate chronic kidney disease (CKD2-3). In other embodiments the subject has severe chronic kidney disease (CKD4). In other embodiments, the subject is on aspirin or diuretic therapy.

It will be recognized by persons with ordinary skill in the art that patients with gout or at risk of developing gout may be administered agents such as non-steroidal anti-inflammatory drugs (NSAIDS), colchicine, steroids, or similar medicaments to treat or manage gout flares. Accordingly, in certain embodiments of the methods described herein, the subjects may also be administered an agent such as an NSAID, colchicine or a steroid.

The methods described herein may be accomplished by the administration of a compound that generates the compound of Formula (IV) or a salt thereof via a chemical reaction after being administered. Such compounds include prodrugs of the compound of Formula (IV). Prodrugs of a compound are prepared by modifying functional groups present in the compound in such a way that the modifications may be cleaved in vivo to release the parent compound, or an active metabolite. For example, prodrugs include compounds wherein a hydroxy, amino, or sulfhydryl group in a compound is bonded to any group that may be cleaved in vivo to regenerate the free hydroxyl, amino, or sulfhydryl group, respectively. Certain prodrugs may increase the bioavailability of the compounds of the embodiments when such compounds are administered to a subject (e.g., by allowing an orally administered compound to be more readily absorbed into the blood) or which enhance delivery of the parent compound to a certain organ or tissue (e.g., adipose tissue, kidney, liver, muscle or joints) relative to the parent species. More particularly, prodrugs of the compound of Formula (IV) include esters, amides, and carbamates (e.g., N,N-dimethylaminocarbonyl) of the hydroxy functional group of the compound of Formula (IV). The compounds of Formulae (I), (II) and (III) are non-limiting examples of prodrugs of the compound of Formula (IV). Further examples of prodrugs can be found in J. Rautio et al. Prodrugs: design and clinical applications, Nat. Rev. Drug Discov., 7, 255-270 (2008); Edward B. Roche, ed., Bioreversible Carriers in Drug Design, American Pharmaceutical Association and Pergamon Press, (1987); and T. Higuchi and V. Stella, Pro-drugs as Novel Delivery Systems, Vol. 14 of the A.C.S. Symposium Series (1975), each of which are hereby incorporated by reference herein.

The compounds disclosed herein are contemplated to exhibit therapeutic activity when administered in an amount which can depend on the particular case. The variation in amount can depend, for example, on the subject being treated and the active ingredients chosen. A broad range of doses can be applicable. Dosage regimes may be adjusted to provide the optimum therapeutic response. For example, several divided doses may be administered daily, weekly, monthly or other at suitable time intervals or the dose may be proportionally reduced as indicated by the exigencies of the situation. Such dosages are optionally altered depending on a number of variables, not limited to the activity of the one or more active ingredients used, the disease or condition to be treated, the mode of administration, the requirements of the individual subject, the severity of the disease or condition being treated, and the judgment of the practitioner.

Depending on factors such as the diagnosis, symptoms, and therapeutic goals of a particular subject, a wide range of dosages of the compound of Formula (I), (II), (III) or (IV) or a pharmaceutically acceptable salt thereof can be contemplated. In various embodiments, the compound may be administered from about 10 mg to about 1000 mg per day. For example, (−)-halofenate, (−)-halofenic acid, or a pharmaceutically acceptable salt thereof may be administered at about 50 mg/day, about 100 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

Dose titration or dose escalation protocols may be employed to determine the proper or optimal dose to administer to a subject. For example, dose titration or escalation studies may select for doses that improve efficacy or tolerability. Dose titration or escalation allows for the gradual adjusting of the dose administered until the desired effect is achieved. Dose titration gradually decreases the dosage administered while dose escalation gradually increases the dose administered. Methods of dose titration and escalation are well known in the art. As a non-limiting example, a subject may be administered 200 mg/day halofenate, halofenic acid, or a pharmaceutically acceptable salt thereof every day and measured for serum uric acid levels on a daily basis. The dosage may be increased or decreased, for example, on a weekly basis. The subject may be monitored for a period of, for example, 2 to 12 weeks to find the desired dose.

Compounds of Formula (I), (II), (III) or (IV) or a pharmaceutically acceptable salt thereof can be incorporated into a variety of formulations and medicaments for therapeutic administration. More particularly, these compounds can be formulated into pharmaceutical compositions by combination with appropriate, pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, or intratracheal administration. Moreover, the compound can be administered in a local rather than systemic manner, in a depot or sustained release formulation. In addition, the compounds can be administered in a liposome.

Compounds of Formula (I), (II), (III) or (IV) or a pharmaceutically acceptable salt thereof can also be formulated with common excipients, diluents or carriers and compressed into tablets, or formulated as elixirs or solutions for convenient oral administration, or administered by the intramuscular or intravenous routes. The compounds can be administered transdermally, and can be formulated as sustained release dosage forms and the like. In one embodiment, the above methods may further comprise the administration of a second urate-lowering agent selected from the group consisting of a xanthine oxidase inhibitor, an inhibitor of uric acid production, a uricosuric agent and a uricase. In one embodiment, the method comprise administering a pharmaceutical composition comprising a first urate-lowering agent and a second therapeutic agent, as described herein, to a subject whose serum uric acid level is at least about 4 mg/dL, at least about 5 mg/dL, at least about 6 mg/dL, at least about 6.8 mg/dL, at least about 7 mg/dL, at least about 8 mg/dL, at least about 9 mg/dL, at least about 10 mg/dL, or at least about 11 mg/dL. The amount of decrease of serum uric acid level that is appropriate may vary depending on the subject, depending upon the subject's overall medical condition. Similarly, the amount of decrease of serum uric acid level that is appropriate for one group of subjects sharing a common medical condition may be different from that which is appropriate for a different group of subjects sharing a different medical condition. In a particular embodiment, the application discloses combination therapy and methods of concomitant administration of a first and second urate-lowering agent (wherein these first and second urate-lowering agents are described herein). Combination therapy and concomitant administration refer to the administration of the two agents (i.e., a first agent and a second urate-lowering agent, as described herein) in any manner in which the pharmacological effects of both are manifested in the subject at the same time. Thus, such administration does not require that a single pharmaceutical composition, the same type of formulation, the same dosage form, or even the same route of administration be used for administration of both the first and second urate-lowering agents, or that the two agents be administered at the same time. Such administration may be accomplished most conveniently by the same dosage form and the same route of administration, at substantially the same time. For example, a first urate-lowering agent, e.g. halofenate, halofenic acid, or a pharmaceutically acceptable salt thereof, and a second urate-lowering agent, e.g. xanthine oxidase inhibitor (e.g., allopurinol or febuxostat), can be administered to the human subject together in a single oral dosage composition, such as a tablet or capsule, or each agent can be administered in separate oral dosage formulations. One advantage with separate formulations is an added flexibility in dosing, i.e. the dosage of the first and second urate-lowering agents can be changed independently, quickly, and easily. Where separate dosage formulations are used, the first and second urate-lowering agents can be administered at essentially the same time (i.e., simultaneously or concurrently), or at separately staggered times (i.e., sequentially). In another embodiment, the second urate-lowering agent is a xanthine oxidase inhibitor, preferably selected from the group consisting of allopurinol, febuxostat, oxypurinol, tisopurine, inositol, phytic acid, myo-inositiol, kaempferol, myricetin, and quercetin, especially allopurinol or febuxostat. In yet another embodiment, the second urate-lowering agent is allopurinol and is administered at from about 50 mg to about 800 mg per day. In another embodiment, the first urate-lowering agent is (−)-halofenate and is administered at from about 100 mg to about 600 mg per day, and the second urate-lowering agent is febuxostat and is administered at from about 40 mg to about 120 mg per day. In another embodiment, the second urate-lowering agent is a uricosuric agent, preferably selected from the group consisting of probenecid, 2-((5-bromo-4-(4-cyclopropylnaphthalen-1-yl)-4H-1,2,4-triazol-3-yl)thio)acetic acid, potassium 4-(2-((5-bromo-4-(4-cyclopropylnaphthalen-1-yl)-4H-1,2,4-triazol-3-yl)thio)acetamido)-3-chlorobenzoate, RDEA684, benzbromarone, sulfinpyrazone, amlodipine, atorvastatin, fenofibrate, guaifenesin, losartan, adrenocorticotropic hormone and cortisone, especially probenecid.

The pharmaceutical compositions of the present disclosure may be administered once daily (QD), twice daily (BID), three times daily (TID) or four times per day (QID). In one embodiment, the composition of the present disclosure is administered once daily (QD). In another embodiment, the composition of the present disclosure is administered twice daily (BID). Particular embodiments covering compositions, formulations and their method of uses are disclosed in a PCT Patent Application No. PCT/US 11/59394 entitled “Methods for Treating Hyperuricemia in Patients with Gout Using Halofenate or Halofenic Acid and a Second Urate-Lowering Agent” filed Nov. 4, 2011, and the PCT Application is incorporated herein in its entirety.

### EXAMPLES

### Example 1

### Clinical Trial Results and Analyses Showing Serum Uric Acid Lowering in Subjects with CKD

FIGS. 1-4 were generated based on the pooled analysis of four phase 2 studies conducted with (−)-halofenate in type 2 diabetic patients. In these studies a total of 955 patients were enrolled. In the M102-20303 study, two dose levels of (−)-halofenate (200 mg and 400 mg) or placebo were given orally daily to a total of 217 patients for 12 weeks. In the M102-20405 study, (−)-halofenate at a dose of 600 mg or placebo was given daily to a total of 100 patients for 12 weeks. In the M102-20509 study, three dose levels of (−)-halofenate (200 mg, 400 mg and 600 mg), Actos® 30 mg or placebo were given orally daily to a total of 396 patients for 16 weeks. In M102-20814 study, two dose levels of (−)-halofenate (400 mg and 600 mg), Actos® 30-45 mg or placebo were given orally daily to a total of 242 patients for 24 weeks. In these studies, change in serum uric acid (sUA) from baseline was one of the important endpoints. In order to maintain the blinded status, the sUA results following administration of double-blind study medication were not available to any parties until the studies were unblinded. Patients who added or changed the existing doses of medications that are known to influence serum uric acid levels were excluded from the serum uric acid analysis. Patients received either (−)-halofenate or matching placebo during the study were included in this exploratory analysis. Patients who received (−)-halofenate during the study but had no measurable (−)-halofenate in the blood were defined as non-compliant and were excluded from the analysis. This analysis indicated statistically significant dose dependent reduction in serum uric acid from baseline: 2% in the placebo group (n=252), −11% in the 200 mg group (n=125), −20% in the 400 mg group (n=174), and −27% in the 600 mg group (n=159); p<0.0001 for all dose groups. In order to evaluate the sUA response in CKD patients, in addition to the analysis described above, the patients were subdivided based on their baseline kidney function status. The kidney function status of each patient was evaluated by estimating the creatinine clearance (glomerular filtration rate) using the Cockcroft-Gault equation [Cockcroft-Gault GFR=(140−age)×(Body weight in kg)×(0.85 if female)/(72×serum creatinine)]. For estimating the baseline creatinine clearance, baseline body weight (kg) and baseline serum creatinine values were used and similarly, for estimating the end-of-the study creatinine clearance, end-of-study body weight and end-of-study serum creatinine values were used. Based on the estimated creatinine clearance values (GFR) patients were divided into five CKD groups—Normal kidney function (CKD0)—GFR≧120 mL/min; CKD1—GFR of 90 to 119 mL/min; CKD2 (mild CKD)—GFR of 60 to 89 mL/min; CKD3 (moderate CKD)—GFR of 30 to 59 mL/min; CKD4 (severe CKD)—GFR of 15 to 29 mL/min; CKD5 (kidney failure)—GFR less than 15 mL/min. In this analysis there were no patients with CKD4 (severe CKD) or CKD5 (kidney failure).

As shown in FIG. 1 and Table 1, data from the pooled analysis of phase 2 studies demonstrates that the treatment with (−)-halofenate at a daily dose of 600 mg reduces the serum uric acid significantly from baseline in patients with all stages of CKD tested. The mean sUA changes from baseline were −25% in the Normal and CKD1 patients with a mean GFR of 119 mL/min (n=105), −28% in the CKD2 patients with a mean GFR of 75 mL/min (n=48), and −36%, in the CKD3 patients with a mean GFR of 52 mL/min (n=6).

TABLE 1

| | | Mean | | | |
|---|---|---|---|---|---|
| | | Baseline | Mean | | |
| | | estimated | sUA at | Mean | |
| (−)-halofenate | | Cr Cl | Baseline | Change in | p- |
| 600 mg | N | (mL/min) | (mg/dL) | sUA (%) | Value |
| Normal and CKD1 | 105 | 119.4 | 4.9 | −25.3% | <0.0001 |
| CKD2 (mild CKD) | 48 | 74.7 | 4.9 | −28.2% | <0.0001 |
| CKD3 (moderate | 6 | 51.8 | 6.2 | −35.6% | 0.0002 |
| CKD) | | | | | |

As shown in FIG. 2 and Table 2, data from the analysis described above of phase 2 studies demonstrates that treatment with (−)-halofenate reduces serum uric acid in dose-dependent manner in CKD2 and CKD3 patients; the sUA changes from baseline in Stage 2 and Stage 3 CKD patients (eGFR <90 and <60 ml/min, respectively) were −14% in the 200 mg group (n=43), −21% in the 400 mg group (n=74), and −29%, in the 600 mg group (n=54).

TABLE 2

| | | Mean | | | |
|---|---|---|---|---|---|
| | | Baseline | Mean | | |
| | | estimated | sUA at | Mean | |
| | | Cr Cl | Baseline | Change in | p- |
| | N | (mL/min) | (mg/dL) | sUA (%) | Value |
| (−)-halofenate | 43 | 70.8 | 5.6 | −14.30% | <0.0001 |
| 200 mg | | | | | |
| (−)-halofenate | 74 | 74.1 | 5.2 | −21.30% | <0.0001 |
| 400 mg | | | | | |
| (−)-halofenate | 54 | 72.1 | 5 | −29.00% | <0.0001 |
| 600 mg | | | | | |

FIG. 3 is a scatter plot with linear regression analysis where baseline estimated creatinine clearance is plotted on the x-axis and the percent change in serum uric acid is plotted on the y-axis. This data demonstrates that the change in serum acid is independent of baseline CKD status (R²=0.009201) as similar changes in serum uric acid was observed across all CKD stages tested.

Referring to FIG. 4 and Table 3, data from the pooled analysis of phase 2 studies described above demonstrates that treatment with (−)-halofenate at daily doses of 200 mg, 400 mg, and 600 mg does not result in deterioration of kidney function status compared to the placebo group; the changes in estimated creatinine clearance from baseline were −2.4 mL/min in the placebo group (n=252), −1.4 mL/min in the 200 mg group (n=125), −0.5 mL/min in the 400 mg group (n=174), and −2.7 mL/min in the 600 mg group (n=159). The changes in estimated creatinine clearance values in the 200 mg, 400 mg, and 600 mg groups compared to placebo group were not statistically significant.

TABLE 3

| | | Mean | Mean | | |
|---|---|---|---|---|---|
| | | Baseline | Baseline | Change in | |
| | | estimated | estimated | estimated | |
| | | Cr Cl | Cr Cl | Cr Cl | p- |
| | N | (mL/min) | (mL/min) | (mL/min) | Value |
| Placebo | 252 | 104.0 | 101.6 | −2.4 | |
| (−)-halofenate | 125 | 103.8 | 102.4 | −1.4 | 0.5688 |
| 200 mg | | | | | |
| (−)-halofenate | 174 | 103.9 | 103.5 | −0.5 | 0.2508 |
| 400 mg | | | | | |
| (−)-halofenate | 159 | 103.4 | 100.6 | −2.7 | 0.8303 |
| 600 mg | | | | | |

### Example 2

### Clinical Trial Results and Analyses Showing Lowering of HbA1c, Fasting Plasma Glucose, and Triglycerides in Subjects with CKD

FIGS. 5-7 were generated based on a randomized, double-blind, placebo-controlled phase 2 study, M102-20303. In this study, two dose levels of (−)-halofenate (200 mg and 400 mg) or placebo were given orally daily for 12 weeks to a total of 217 type 2 diabetes patients who were inadequately controlled on existing insulin therapy. In addition to their stable dose of insulin, these patients received the study drug for 12 weeks, and followed up for an additional four weeks. In this study, change in HbA1c from baseline at week 16 was the primary endpoint and changes in fasting plasma glucose and triglyceride from baseline at Week 12 were secondary endpoints. In order to maintain the blinded status, the HbA1c results following administration of double-blind study medication were not available to any parties until the studies were unblinded. In this study, patients were not allowed to add any other glucose lowering agent(s) or to change their existing doses of insulin. Patients who added lipid lowering agent(s) or changed their existing doses of lipid lowering agent(s) were excluded from the triglyceride analysis. Patients who received (−)-halofenate during the study but had no measurable (−)-halofenate in the blood were defined as non-compliant and were excluded from the analysis. In this study, treatment with both doses of (−)-halofenate resulted in statistically significant reductions from baseline in HbA1c (−0.9% and −1.0% on 200 mg and 400 mg, respectively, vs.-0.3% on Placebo (insulin alone), p=0.002). Treatment with (−)-halofenate also resulted in a dose-dependent decrease in FPG from baseline (mean observed changes at Week 12 were 8.9 mg/dL, −10.9 mg/dl, and −29.2 mg/dL for the Placebo, 200-mg, and 400-mg groups, respectively). The change in triglycerides from baseline to Week 12 was a 27.5 mg/dL (13.4%) increase in the Placebo group vs. a 15.9 mg/dL (9.3%) increase in the 200 mg group, and a 13.0 mg/dL (6.7%) decrease in the 400 mg group (p=0.074).

Referring to FIG. 5 and Table 4, data from the above mentioned phase 2 study, M102-20303, demonstrates that the treatment with (−)-halofenate at a daily dose of 400 mg for 12 weeks reduces the HbA1c level in CKD2 and CKD3 patients (mild to moderate CKD, n=22) significantly compared to the similar group of CKD patients treated with placebo (insulin only, n=21). The observed changes in HbA1c were −1.1% and −0.3% in mild to moderate CKD patients treated with (−)-halofenate 400 mg and mild to moderate CKD patients treated with placebo, respectively (p=0.0459).

TABLE 4

| CKD2 and CKD3 | | Baseline | End-of- | Change in | |
|---|---|---|---|---|---|
| (Mild to moderate | | HbA1c | study | HbA1c | p- |
| CKD) | N | (%) | HbA1c (%) | (%) | Value |
| Placebo (insulin | 21 | 9.4 | 9.1 | −0.3 | |
| only) | | | | | |
| (−)-halofenate | 22 | 9.2 | 8.1 | −1.1 | 0.0459 |
| 400 mg | | | | | |

Referring to FIG. 6 and Table 5, data from the above mentioned phase 2 study, M102-20303, demonstrates that the treatment with (−)-halofenate at a daily dose of 400 mg for 12 weeks reduces the FPG level in CKD2 and CKD3 patients (mild to moderate CKD, n=22) significantly compared to the similar group of CKD patients treated with placebo (insulin only, n=19). The observed changes in FPG were −18.0% and 7.0% in mild to moderate CKD patients treated with (−)-halofenate 400 mg and mild to moderate CKD patients treated with placebo, respectively (p=0.0325).

TABLE 5

| CKD2 and CKD3 | | Baseline | End-of- | | |
|---|---|---|---|---|---|
| (Mild to moderate | | FPG | study | Change in | p- |
| CKD) | N | (mg/dL) | FPG (mg/dL) | FPG (%) | Value |
| Placebo (insulin | 19 | 167.0 | 177.0 | 7.8 | |
| only) | | | | | |
| (−)-halofenate | 22 | 182.0 | 136.5 | −17.6 | 0.0325 |
| 400 mg | | | | | |

Referring to FIG. 7 and Table 6, data from the above mentioned phase 2 study, M102-20303, demonstrates that the treatment with (−)-halofenate at a daily dose of 400 mg for 12 weeks reduces the triglyceride level in CKD2 and CKD3 patients (mild to moderate CKD, n=22) compared to the similar group of CKD patients treated with placebo (insulin only, n=20). The observed changes in triglyceride were −33.6 mg/dL and 1.4 mg/dL in mild to moderate CKD patients treated with (−)-halofenate 400 mg and mild to moderate CKD patients treated with placebo, respectively (p=0.1757).

TABLE 6

| | | Baseline | End-of-study | Change in | |
|---|---|---|---|---|---|
| CKD2 and CKD3 | | triglyc- | triglyc- | triglyc- | |
| (Mild to moderate | | eride | eride | eride | p- |
| CKD) | N | (mg/dL) | (mg/dL) | (mg/dL) | Value |
| Placebo (insulin | 20 | 184.0 | 185.4 | 1.4 | |
| only) | | | | | |
| (−)-halofenate | 22 | 202.2 | 168.6 | −33.6 | 0.1757 |
| 400 mg | | | | | |

### Example 3

### Clinical Trial Results and Analyses Showing Serum Uric Acid Lowering in Subjects on Aspirin and Diuretic Therapies

FIGS. 7-8 were generated based on the pooled analysis described in Example 1.

Referring to FIG. 7, data from the pooled analysis of phase 2 studies described above demonstrates that treatment with (−)-halofenate at daily doses of 200 mg, 400 mg, and 600 mg does not result in clinically significant loss of the urate-lowering effect when co-administered with low to medium doses of aspirin (less than or equal to about 325 mg/day) compared to the (−)-halofenate groups at equivalent doses not receiving aspirin. The changes in serum uric acid (sUA) from baseline in aspirin treated patients were −13% in the 200 mg group (n=45), −20% in the 400 mg group (n=57), and −27% in the 600 mg group (n=55); the changes in serum uric acid (sUA) from baseline in patients not taking aspirin were −10% in the 200 mg group (n=80), −20% in the 400 mg group (n=117), and −26% in the 600 mg group (n=104). The changes in uric acid between the aspirin treated and aspirin un-treated groups at the 200 mg, 400 mg, and 600 mg groups were not statistically different.

Referring to FIG. 8, data from the pooled analysis of phase 2 studies described above demonstrates that treatment with (−)-halofenate at daily doses of 200 mg, 400 mg, and 600 mg does not result in clinically significant loss of the urate-lowering effect when co-administered with thiazide or other diuretics compared to the (−)-halofenate groups at equivalent doses not receiving any diuretics. The changes in serum uric acid (sUA) from baseline in thiazide diuretic treated patients were −16% in the 200 mg group (n=11), −17% in the 400 mg group (n=16), and −24% in the 600 mg group (n=28); the changes in serum uric acid (sUA) from baseline in patients taking any diuretics were −12% in the 200 mg group (n=17), −15% in the 400 mg group (n=25), and −24% in the 600 mg group (n=34); the changes in serum uric acid (sUA) from baseline in patients not taking any diuretics were −11% in the 200 mg group (n=108), −21% in the 400 mg group (n=149), and −27% in the 600 mg group (n=125). The changes in uric acid between the diuretic treated and diuretic un-treated groups at the 200 mg, 400 mg, and 600 mg groups were not statistically different.

While the foregoing description describes specific embodiments, those with ordinary skill in the art will appreciate that various modifications and alternatives can be developed. Accordingly, the particular embodiments and examples described above are meant to be illustrative only, and not to limit the scope of the invention, which is to be given the full breadth of the appended claims, and any and all equivalents thereof.

What is claimed is:

1. A method of lowering serum uric acid level in a subject with impaired renal function, comprising administering to the subject a compound of Formula (I) 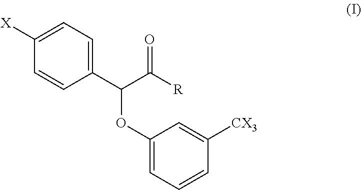
wherein R is selected from the group consisting of hydroxy, lower aralkoxy, di-lower alkylamino-lower alkoxy, lower alkanamido-lower alkoxy, benzamido-lower alkoxy, ureido-lower alkoxy, N′-lower alkyl-ureido-lower alkoxy, carbamoyl-lower alkoxy, halophenoxy-substituted lower alkoxy, carbamoyl-substituted phenoxy, carbonyl-lower alkylamino, N,N-di-lower alkylamino-lower alkylamino, halo-substituted lower alkylamino, hydroxyl-substituted lower alkylamino, lower alkanolyloxy-substituted lower alkylamino, ureido, and lower alkoxycarbonylamino; and each X is independently a halogen; or a pharmaceutically acceptable salt thereof.

2. The method of claim 1, wherein the compound or a pharmaceutically acceptable salt thereof is (−)-halofenate or (−)-halofenic acid, or a pharmaceutically acceptable salt thereof.

3. The method of claim 1, wherein the compound (−)-halofenate.

4. A method of treating a subject having a condition associated with an elevated serum uric acid level and with impaired renal function, comprising administering to the subject a compound of Formula (I) 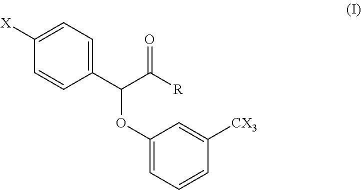
wherein R is selected from the group consisting of hydroxy, lower aralkoxy, di-lower alkylamino-lower alkoxy, lower alkanamido-lower alkoxy, benzamido-lower alkoxy, ureido-lower alkoxy, N′-lower alkyl-ureido-lower alkoxy, carbamoyl-lower alkoxy, halophenoxy-substituted lower alkoxy, carbamoyl-substituted phenoxy, carbonyl-lower alkylamino, N,N-di-lower alkylamino-lower alkylamino, halo-substituted lower alkylamino, hydroxyl-substituted lower alkylamino, lower alkanolyloxy-substituted lower alkylamino, ureido, and lower alkoxycarbonylamino; and each X is independently a halogen; or a pharmaceutically acceptable salt thereof.

5. The method of claim 4, wherein the compound or a pharmaceutically acceptable salt thereof is (−)-halofenate or (−)-halofenic acid, or a pharmaceutically acceptable salt thereof.

6. The method of claim 4, wherein the compound is (−)-halofenate.

7. The method of claim 4, wherein the condition associated with an elevated serum uric acid level is gout.

8. The method of claim 5, wherein the condition is acute gout, chronic gout, moderate gout, refractory gout or severe gout.

9. A method for the treatment of hyperuricemia in a subject with gout comprising administering to the subject in need thereof a compound of Formula (I) of claim 1 or a pharmaceutically acceptable salt thereof, wherein the subject has impaired renal function.

10. The method of claim 9, wherein the compound of Formula (I) of claim 1 or a pharmaceutically acceptable salt thereof is a compound of Formula (II) 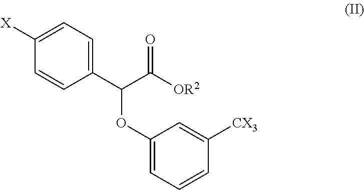
wherein R² is a selected from the group consisting of phenyl-lower alkyl, lower alkanamido-lower alkyl, and benzamido-lower alkyl, and each X is independently a halogen; or a pharmaceutically acceptable salt thereof.

11. The method of claim 9, wherein the compound or a pharmaceutically acceptable salt thereof is (−)-halofenate or (−)-halofenic acid, or a pharmaceutically acceptable salt thereof.

12. The method of claim 9, wherein the compound is (−)-halofenate.

13. The method of claim 9, wherein the impaired renal function is chronic kidney disease.

14. The method of claim 13, wherein the chronic kidney disease is mild or moderate.

15. The method of claim 13, wherein the chronic kidney disease is severe.

16. The method of claim 9, wherein the compound is administered at a dose that is independent of the stage of chronic kidney disease.

17. The method of claim 9, wherein the administration of the compound results in no clinically significant adverse effect on renal function.

18. The method of claim 9, wherein the administration of the compound results in a lowering of HbA1c, blood glucose, or fasting plasma glucose levels.

19. The method of claim 9, wherein the administration of the compound results in a lowering of triglyceride levels.

20. The method of claim 9, wherein the compound is (−)-halofenate and the compound is administered at from about 100 mg to about 1000 mg per day.

21. The method of claim 9, wherein the subject is undergoing aspirin or a diuretic therapy.

22. The method of claim 7, further comprising the administration of a second urate-lowering agent selected from the group consisting of a xanthine oxidase inhibitor, an inhibitor of uric acid production, a uricosuric agent and a uricase.

23. The method of claim 7, wherein the second urate-lowering agent is selected from the group consisting of allopurinol, febuxostat, ulodesine, forodesine, oxypurinol, tisopurine, inositol, phytic acid, myo-inositiol, kaempferol, myricetin, quercetin, probenecid, 2-((5-bromo-4-(4-cyclopropylnaphthalen-1-yl)-4H-1,2,4-triazol-3-yl)thio)acetic acid, potassium 4-(2-((5-bromo-4-(4-cyclopropylnaphthalen-1-yl)-4H-1,2,4-triazol-3-yl)thio)acetamido)-3-chlorobenzoate, RDEA684, benzbromarone, sulfinpyrazone, amlodipine, atorvastatin, fenofibrate, levotofisopam, guaifenesin, losartan, adrenocorticotropic hormone and cortisone.

24. The method of claim 23, wherein second urate-lowering agent is febuxostat.
